(19) United States
(12) Patent Application Publication
(10) Pub. No.: US 2010/0068745 A1
(43) Pub. Date: Mar. 18, 2010

(54) PROCESS AND KIT FOR MEASURING THE CONDITION OF THE FIBRINOLITIC SYSTEM

(75) Inventors: Andras Berta, Debrecen (HU); Adrienne Csutak, Debrecen (HU); Tamas Sperka, Debrecen (HU); Jozsef Tozser, Debrecen (HU)

Correspondence Address: LADAS & PARRY LLP, 26 WEST 61ST STREET, NEW YORK, NY, 10023, US

(73) Assignee: DEBRECENI EGYETEM

(21) Appl. No.: 12/474,705

(22) Filed: May 29, 2009

Related U.S. Application Data
(63) Continuation-in-part of application No. PCT/HU2007/000116, filed on Nov. 30, 2007.

(30) Foreign Application Priority Data
Nov. 30, 2006 (HU) P 0600891

(51) Int. Cl. C12Q 1/34; C12M 1/34

(52) U.S. Cl. 435/18; 435/288.4

(57) ABSTRACT

The invention relates to an assay and a kit for the determination of the status of the fibrinolytic system. In particular the invention relates to a method for the determination of the status of the fibrinolytic system from tear fluid samples. Additionally the invention relates to a sterilized, calibrated capillary equipped with a plastic balloon, suitable for tear fluid sampling, and a kit for the assessment of the status of the fibrinolytic system, or for the determination of its balance, comprising a sampling capillary, reaction sheets, color scale to help the evaluation, and reagents

The invention relates to an assay and a kit for the determination of the status of the fibrinolytic system. In particular the invention relates to a method for the determination of the status of the fibrinolytic system from tear fluid samples.

In many processes taking place in the anterior segment of the eye the cellular, viral and bacterial proteases play an essential role. In some cases, as a result of the protease hyperfunction, harmful degradative processes may take place, occasionally leading even to blindness, and in some other cases the low protease activity occurring as a result of inappropriate expression, or the increased appearance of protease-inhibitors may have harmful effects, i.e. causing unsatisfactory wound healing.

Protease hyperfunction may occur in case of bacterial and viral ocular infections (i.e. conjunctivitises), and in other processes affecting the cornea (i.e. keratitis, ulcus).

Protease deficiency may primarily occur after the laser refraction operation, widely used nowadays. Numerous types of the surgical interventions are known—photorefractive keratectomy (PRK), laser “in situ” keratomyleusis (LASIK) and laser assisted subepithelial keratomyleusis (LASEK)—which target the correction of the corneal refraction deficiencies, by “re-profiling the surface of the cornea. In case of wound healings with complications following laser treatment corneal stromal haze may occur, which, in some cases reduces even the corrected vision acuity. Millions of patients have vision correction operation in each year. Within a few months following the operation 8-10% of the patients report blurred vision acuity.

Biochemical studies support the significance of the tear fluid in the corneal wound healing processes, which, according to our knowledge is controlled by two big systems through activators and inhibitors. The significance of the first, so called plasminogen activator/plasmin system is based on its role in the degradation and removal of the damaged extracellular matrix.

The other system, on the basis of the activated keratocytes, is responsible for the generation of the collagen fibrils newly synthesized to replace the damaged collagen structures. But at the same time the two systems are not independent from each other, because in most cases the activation of the proenzyme form of the collagenases responsible for the decomposition of the collagen to their active form is carried out by the plasmin generated by the plasminogen activators. Correct function of the systems described above is necessary for the re-epithelisation. If the balance of the two systems breaks for any reason, the final outcome may be cicatrisation or long lasting wound healing, or even corneal ulcer.

Within 24 hours following de-epithelisation polymorphonuclear granulocytes appear in the stroma, which migrate into the stroma from the tear film. The plasminogen level of the tear film increases and by turning on the plasminogen activator/plasmin system removal of the tissue and cell debris, and repair of the damaged collagen and extracellular matrix begin. From the aspects of the regeneration of the epithelium removal of the generated tissue and cellular debris is useful and desirable, but at the same time the increased synthesizing activity of the stromal keratinocytes may result in formation of scars and this way formation of stromal haze, and as a consequence of this—after the laser surgery intervention on the cornea—the achieved refraction effect may weaken. The other side of the proteolytic system, formation of the corneal ulcers, which—independently from the etiology—is the enzymatic decomposition of the basic matrix of the cornea, and this is why clarification of the role of the proteolytic enzymes in the processes of the anterior segment of the eye is an important task, both from pathogenetic and diagnostics aspects. Although the enzymatic mechanism of the disintegration of the cornea is widely accepted I there are only limited data available in the literature about the exact role of the individual enzymes.

Nowadays the number of the photorefractive laser surgery intervention on the cornea is exponentially growing. The corneal wound healing processes are practically affecting a wide scale of the opthalmologic operations. The practicing ophthalmologists are confronted day by day with the inflammatory problems affecting the anterior segment of the eye. Beyond that clarification of the problems connected to this may help the development of therapeutic procedures more effective than the existing ones.

From the state of art many methods are known for the detection and determination of the components found in the tear fluid.

In the U.S. Pat. No. 5,352,411 an equipment and procedure is described for the determination of the components of the tear fluid. Owing to this solution it is possible to detect and determine the organic and ionic components of the tear fluid.

In the U.S. Pat. No. 7,121,666 an equipment and procedure is described for the measurement of the stability of the tear fluid. According to this solution pictures are prepared, on the basis of which the stability of the tear fluid can be deduced from the dispersion pattern of the tear film.

In the patent application No. U.S. a procedure is described for the determination of fluorescein in the tear films. In the course of the procedure a fluorescein-containing coloring agent is introduced to the tear film, and as a result of this the occasional abnormalities of the tear film can be determined with higher sensitivity and specifity, which abnormalities are related to the ocular irritation and eye surface diseases of the patient.

The methods recently used for the determination of the plasminogen activator are the following. Semi-quantitative determination of the activity is possible by dropping the sample into casein sheets containing fibrinogen. In such cases the size of the received lysis zone gives information about the enzymatic activity. Equipment is not needed for this method of measurement, but detection must be carried out in a humid chamber, technically difficult to execute, and needs 18-24 hours' reaction time and requests a distinct gel staining procedure. Direct measurement of the urokinase activity with specific chromogenic substrate (for example piroGlu-Gly-Arg-pNA) can also be realized with. a similarly long reaction time, which needs photometric detection. The indirect chromogenic substrate procedure based on the measurement of plasmin activity can be realized with a significantly shorter (2-4 hours) incubation' time, but to this microtiter incubation in a humid chamber is needed and a microtiter reader, or incubation in a closed tube, and a photometric equipment suitable for the measurement of small volumes. Originally this method was described for measurements in Eppendorf tubes (Shimada et al., 1981), and this proved to be suitable for the measurement of the plasminogen activity of tear fluid samples (Tözsér et al., 1989).

But at the same time no solution is known which is routinely useful in the practice, and allows a quick, diagnostic measurement from tear fluid samples. In particular no method is known which would be suitable for the quick measurement of the plasminogen activator level of the tear fluid samples, without special equipment. Up to know no uniformly accepted tear fluid sampling method was developed, although it is known, that the way of sampling the tear fluid significantly influences the results of the measurement. Moreover, up to now doctors don't have the possibility to indicate in advance that which patients will develop corneal stromal haze following laser treatment. Detection of the absence of enzymatic activity may indicate the complication in advance. Supplementation of the missing enzyme may inhibit the development of the haze.

As a consequence of what was mentioned above, and because of the lately considerably spread antibiotic resistance, occurring as a consequence of the protease hyperfunction new therapeutic intervention possibilities are requested, and a diagnostic method suitable for monitoring the activity would be of fundamental importance. In parallel with this there is a great demand for methods which are suitable for solving the problems detailed above, or predicting them exactly and reproducibly.

According to this the invention relates to an assay, which is suitable for the determination of the status of the fibrinolytic system, or for the determination of its balance. In the assay tear fluid sample is used for the determination of the status of the fibrinolytic system, or for the determination of its balance.

This invention can be considered essentially as the improvement of the method mentioned above (Tözsér et al., 1989), which improvement gives the possibility to use significantly smaller volumes (10 μl) than the volumes currently used (100-500 μl), to use much shorter reaction time (20-30 minutes instead of 2-24 hours), and for the evaluation of the results independently from the equipment.

A great advantage of this invention is that the application field of the assay and kit for the determination of the balance of the fibrinolytic system in a short period of time allows the development of therapeutic methods which are much widely usable and individually tailored, compared to the supplementation of the uPA deficiency mentioned above.

A further significant advantage of the method of the invention that contrary to the solutions known from the state of art it doesn't need any instrument. That is for example neither the use of incubation chamber nor spectrophotometer is necessary.

### BRIEF DESCRIPTION OF THE DRAWINGS

FIG. 1 shows capillaries equipped with a flexible balloon of use in the method of the invention.

FIG. 2 shows a reaction plate suitable for use in the present invention.

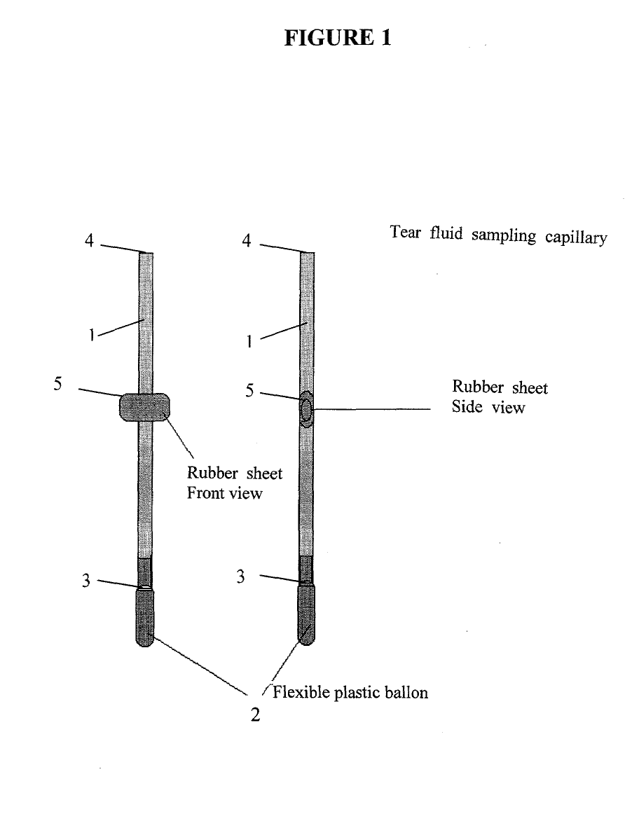

In the course of the method according to the invention a fixed tear fluid sampling protocol is used. In the protocol calibrated and sterilized glass or plastic capillaries are used, equipped with a flexible balloon (FIG. 1), which allows not just the sampling, but also the transfer of the sample into the reaction well. These capillaries can be used not just for sampling, but for the transfer of the reagents into the reaction well. Optionally the necessary reagents can be transferred to the walls of the capillary tubes. Transfer of the reagents can be done by methods well known for the experts, belonging to the state of the arts. According to what is mentioned above the invention relates to the sterilized, calibrated capillary 1, equipped with a flexible plastic balloon. Preferably capillary 1 is calibrated to 5 μl volume. According to the embodiment of the invention the flexible plastic balloon 2 connects to the 3 or 4 end of the capillary 1, and the capillary tube 1 is equipped with a rubber sheet 5. Rubber sheet 5 is suitable for controlling the amount of the tear fluid sample sucked up by the capillary 1.

The method of the invention allows quick determination of the urokinase activity level of the tear fluid samples, and in this procedure evaluation of the results obtained is carried out semi-quantitatively, in harmony with what is mentioned above, without any instrument, particularly without photometric equipment.

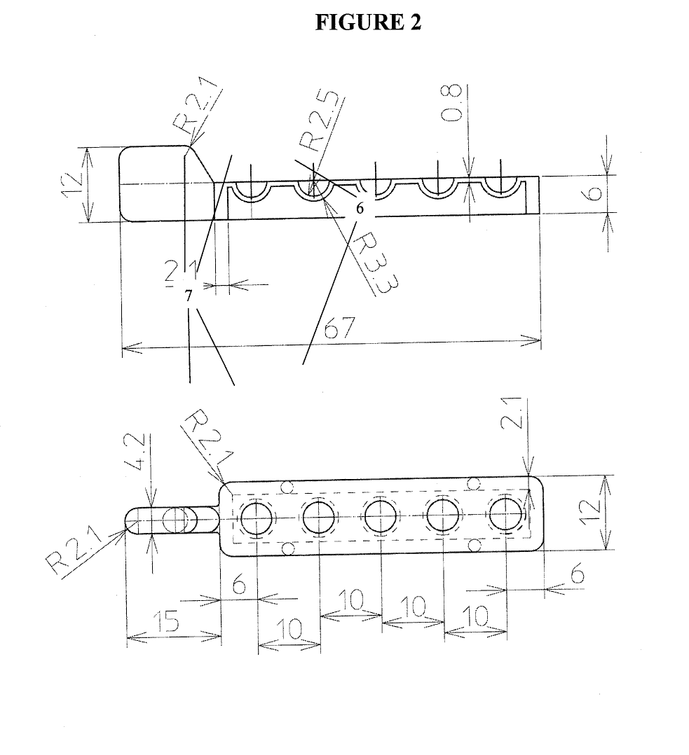

In the course of the evaluation the plastic measuring plates 6 (FIG. 2) are used, which have small volume (20 μl) wells 7, these are suitable for executing small volume color reactions and for semi-quantitative evaluation, and later they can be used for other tear fluid diagnostic measurements.

Again, in harmony with what was mentioned above, the invention relates to the sampling capillaries, reaction plates, the color scale for the evaluation and the diagnostic kit containing the reaction components in suitable concentration. The primary application of the diagnostic kit according to the invention is the selection of the application demand of the urokinase eye-drops used for preventing the corneal stromal haze, and monitoring of the therapy.

The invention is illustrated with the following, non-limiting example.

### EXAMPLE 1

Sterile, volume calibrated glass capillaries according to the invention are used for collecting tear fluid samples, on which—among others—the necessary 5 μl volume is marked, and have rubber pump at their end. From the capillaries the tear fluid sample gets into a well of the reaction plate with the rubber pump. The tear fluid samples can also be transferred by different methods, i.e. by a suitable capillary pipette. Two reagents should be added to the tear fluid sample. This can be done individually, or mixed, with the capillaries mentioned above, or with automated micropipettes. The ratio of the two reagents, and the composition of the buffers applied are the following: 1./3 microliters 20 mM D-Val-Leu-Lys-p-nitroanilide solution, in 100 mM TRIS-HCI, 300 mM NaCl, pH=8.5 buffer. The strongly basic pH of the buffer is essential for the quick reaction. 2./2 microliters 16 mg/ml (282 CU/ml) plasminogen solution, dissolved in water distilled. The high concentration of the solution is essential for the quick reaction. The two components can be stored frozen or in lyophilized form, they can be part of the diagnostic kit in individually or in mixed form, in the latter case the attached description defines the amount of distilled water to be added.

By testing the system with clinical samples, yellowing takes 20 minutes, and after about 30 minutes the differences become clear-cut.

5 microliters urokinase solution (1 IU/ml, stabilized (containing 0.5 PEG or 1% albumin), diluted in 50 mM phosphate buffer (pH=7.4) is transferred into another well of the reaction plate as positive standard, and in case of the tear fluid sample the addition of the reagent is done according to the description.

As negative control (blind) sample 5 microliters of a blind reagent solution (containing 0.5 PEG or 1% albumin) is transferred into another well of the reaction plate as positive standard, and the addition of the reagent is done according to the description at the tear fluid sample.

The time period between the addition of the reagent to the positive standard, blind sample and tear fluid samples may not be longer than one minute.

The reaction plate is allowed to stand for 30 minutes at room temperature, and the extent of discoloration is determined.

For the evaluation of the results obtained the extent of yellowing is evaluated in relation to the ratio of the yellowness of the standard and blind sample. To help evaluation in the course of the 30 minutes' incubation at room temperature (20° C.), the scale demonstrating the extent of the color complying with the yellownesses obtained with the 0.0; 0.1; 0.5; 1.0; 2.0 and 5.0 IU/ml standard urokinase solutions can be used (this is included in the description of the kit).

By the use of the suitable instrument (i.e. Nanodrop) the exact absorption data can also be determined by the photometric evaluation of the small volume sample.

### Table 1

### Comparative Table about the Plasminogen Activator Activities Measured by the Classic Microliter Method, or by the Method According to the Invention

On reaction plate:

| Measured uPa | Incubation period | Incubation | Absorbance |
|---|---|---|---|
| (IU) | (min) | temperature (° C.) | (A₅₀₅-A_{blind}) |
| 0.025 | 23 | room temperature | 0.435 |
| 0.0125 | 23 | room temperature | 0.063 |
| 0.006 | 23 | room temperature | 0.036 |

On microtiter plate:

| Measured uPa | Incubation period | Incubation | Absorbance |
|---|---|---|---|
| (IU) | (min) | temperature (° C.) | (A₅₀₅-A_{blind}) |
| 0.025 | 300 | room temperature | 0.182 |
| 0.0125 | 300 | room temperature | 0.067 |
| 0.006 | 300 | room temperature | 0.043 |

Composition of the reaction according to the invention on the reaction plate: 3 microliters 20 mM D-Val-leu-lys-pNA solution (in 100 mM Tris, 300 mM NaCl, pH=8.5 buffer), 2 microliters 16 mg/ml (282 CU/ml) plasminogen solution (dissolved in MilliQ water), 5 microliters urokinase standard solution (PBS, pH=7.4). Traditional microtiter plate method, in the wells of a 96 wells' plate: 25 microliters 2.5 mM D-Val-Leu-Lys-pNA solution in PBS buffer, pH=7.4), 25 microliters 0.227 mg/ml (4 CU/ml) plasminogen solution (dissolved in MilliQ wafer), 65 microliters buffer PBS, pH=7.4) and 10 microliters urokinase standard solution (PBS, pH=7.4). Photometric determination of the solutions of the reaction plate is carried out with NanoDrop equipment, while the photometric evaluation of the microtiter plates is carried out with ELISA reader.

### REFERENCES

1) Tözsér J. Berta A, Frank J. Holly: Determination of plasminogen activator activities in normal and pathologica/human tears. The Significance of tear plasminogen activators in the inflammatory and traumatic lesions of the cornea and conjunctiva, Acta Opthalmol. 69, 92 (1990)
2) Tözsér J, Berta A: Plasminogen activator inhibitors in human tears. Acta Opthalmol. 69, 426-431 (1991)
3) Csutak A, Silver D M, Tözsér J, Hassan Z, Berta A: Urokinase type plasminogen activator to prevent haze after photorefractive keratectomy and pregnancy as a risk factor for haze in rabbits. Invest. Opthalmol. Vis. Sci. 45, 1329-1333 (2004) (Impact factor: 4,091)
4) Tözsér J, Berta A, Punyiczki M: Plasminogen activator activity and plasminogen independent amidolytic activity in tear fluid from healthy persons and patients with anterior segment inflammation. Clin. Chim. Act. 183, 323-332 (1989)
5) Csutak A, Tözsér J, Békési L, Hassan Z, Berta A, Silver D M: Plasminogen activator activity in tears after Excimer laser Photorefractive Keratectomy. Invest. Opthalmol. Vis. Sci. 41(12) 3743-7 (2000) (Impact factor: 4,858)
6) Csutak A, Silver O M, Tözsér J, Facsk6 A, Berta A: Plasminogen activator activity and inhibition in rabbit tears after photorefractive Keratectomy. Exp. Eye Res. 77, 675-80 (Impact factor: 1,969)

### LIST OF REFERENCE NUMBERS

1—capillary
2—flexible plastic balloon
3—end of the capillary
4—end of the capillary
5—rubber sheet
6—measurement plate
7—well

1. Method for the evaluation of the fibrinolytic system, for the determination of its balance, wherein as starting material tear fluid is used comprising:
a) collection of the tear fluid samples;
b) the collected tear fluid sample is dropped to the reaction plate;
c) as suitable reagents human or animal plasminogen and plasmin specific chromogenic substrate reagents are added to the fluid samples dropped to the reaction plate, and
d) the urokinase plasminogen activator activity is determined from the discoloration of the reaction plate.

2. Reaction plate according to FIG. 2 for the use in kits suitable for the evaluation of the fibrinolytic system.

3. The reaction plate according to claim 2, where the volume of each well is 20 μl.

4. Kit for the assessment of the status of the fibrinolytic system, or for the determination of its balance, comprising a sampling capillary, reaction plates) according to claim 2, color scale to help the evaluation, and suitable reagents selected from the group consisting of human and animal plasminogen and plasmin specific chromogenic substrate reagents.
